(19) United States
(12) Patent Application Publication
(10) Pub. No.: US 2022/0074824 A1
(43) Pub. Date: Mar. 10, 2022

(54) COLLECTION DEVICE

(71) Applicant: Panasonic Intellectual Property Management Co., Ltd., Osaka (JP)

(72) Inventor: YOSHIKI SASAKI, Osaka (JP)

(21) Appl. No.: 17/529,933

(22) Filed: Nov. 18, 2021

Related U.S. Application Data
(63) Continuation of application No. PCT/JP2020/027081, filed on Jul. 10, 2020.

(30) Foreign Application Priority Data
Aug. 7, 2019 (JP) 2019-145460

(51) Int. Cl. G01N 1/22

(52) U.S. Cl. CPC G01N 1/2205; G01N 2001/2244

(57) ABSTRACT

A collection device includes a container that retains a collection liquid and that has a channel through which intake gas travels above the collection liquid retained, and a rotating body that is provided inside the container and that rotates around a rotation axis extending in a direction intersecting an up-down direction. The rotating body has a first blade that protrudes in a direction intersecting the rotation axis. The first blade has a filter that captures a target object included in the intake gas. The filter moves, along with rotation of the rotating body around the rotation axis, from a position inside the channel to a position immersed in the collection liquid. Thereby, the collection device collects, into the liquid, the target object included in the intake gas.

### BACKGROUND

### 1. Technical Field

The present disclosure relates to collection devices that take in gas containing target objects, such as pathogens, and collect the target objects in the gas into liquids.

### 2. Description of the Related Art

Exhaled air of a patient infected with an infectious virus, such as an influenza virus, conceivably contains the infectious virus. It is conceivable that the infectious virus in the exhaled air of the patient scatters into the atmospheric air in the form of particles (i.e., droplets or droplet nuclei) so as to cause droplet infection or airborne infection.

If the virus in the exhaled air or the atmospheric air can be extracted, the infection route can be clarified. Moreover, by quickly determining the presence or absence of the virus in the exhaled air or the atmospheric air, secondary infections can be prevented.

In the related art, devices for collecting components in exhaled air of humans have been developed (see, for example, Japanese Unexamined Patent Application Publication No. 7-103974). The device according to Japanese Unexamined Patent Application Publication No. 7-103974 includes a helical collection pipe, a recovery bottle coupled to a dripping port of the collection pipe, and a cooling container for cooling the collection pipe and the recovery bottle.

A virus extractor that extracts a virus contained in exhaled air of a patient and provides the virus as a sample for diagnosis or research has also been proposed (see, for example, Japanese Unexamined Patent Application Publication No. 2008-119552). The virus extractor according to Japanese Unexamined Patent Application Publication No. 2008-119552 includes a main body, an exhaled-air inlet for taking in the exhaled air of the patient, a trapper that captures the virus contained in the exhaled air of the patient, and a suction unit that suctions a gas component including the exhaled air of the patient.

### SUMMARY

In the device according to Japanese Unexamined Patent Application Publication No. 7-103974, when a person blows exhaled air into the collection pipe from an end thereof, the exhaled air is cooled while it travels through the collection pipe, and a component in the exhaled air adheres to the inner wall of the collection pipe. Then, when the collection pipe is taken out from a cooler and is warmed, a liquid containing the component adhered to the inner wall accumulates in the recovery bottle. However, in Japanese Unexamined Patent Application Publication No. 7-103974, the process of collecting the component in the exhaled air is not simple since the collection pipe has to be cooled and warmed.

In the virus extractor according to Japanese Unexamined Patent Application Publication No. 2008-119552, the exhaled air of the patient is taken into the exhaled-air inlet having a constricted section, and vapor in the exhaled air is condensed and liquefied by utilizing a decompressed state occurring when the exhaled air is released from an opening in the constricted section. The virus contained in the liquid is introduced to an isolation medium disposed in the trapper. However, it is difficult to reliably capture the virus into the trapper. In addition, it is also disclosed in Japanese Unexamined Patent Application Publication No. 2008-119552 that the liquid containing the virus is extracted by inserting a cylindrical member formed of a permeable fabric between the opening in the constricted section and the trapper. However, the process for isolating the liquid containing the virus involves detaching the permeable fabric and setting the permeable fabric in a centrifuge, and is thus troublesome.

One non-limiting and exemplary embodiment provides a collection device that can easily collect a target object in gas into a liquid.

In one general aspect, the techniques disclosed here feature a collection device. The collection device includes a container that retains a liquid and that has a channel through which intake gas travels above the liquid retained, and a rotating body that is provided inside the container and that rotates around a rotation axis extending in a direction intersecting an up-down direction. The rotating body has a first blade that protrudes in a direction intersecting the rotation axis. The first blade has a filter that captures a target object included in the intake gas. The filter moves, along with rotation of the rotating body around the rotation axis, from a position inside the channel to a position immersed in the liquid. Thereby, the collection device collects, into the liquid, the target object included in the intake gas.

The collection device according to the aspect of the present disclosure can easily collect a target object in gas into a liquid. Additional benefits and advantages of the disclosed embodiments will become apparent from the specification and drawings. The benefits and/or advantages may be individually obtained by the various embodiments and features of the specification and drawings, which need not all be provided in order to obtain one or more of such benefits and/or advantages.

### BRIEF DESCRIPTION OF THE DRAWINGS

FIG. 1 is a perspective view of a collection device according to Embodiment 1;

FIG. 2 is a sectional view taken along line II-II in FIG. 1;

FIG. 3 is a sectional view taken along line III-III in FIG. 1;

FIG. 4 is a sectional view illustrating a collection device according to Embodiment 2, as viewed from the left;

FIG. 5 is a sectional view illustrating the collection device according to Embodiment 2, as viewed from the front;

FIG. 6 is a sectional view illustrating a collection device according to Embodiment 3, as viewed from the left;

FIG. 7 is a side view illustrating a collection device according to Embodiment 4, as viewed from the left; and

FIG. 8 is a side view illustrating a collection device according to Embodiment 5.

### DETAILED DESCRIPTION

Embodiments will be described below with reference to the drawings.

It should be noted that a collection device according to the disclosure is not limited to the embodiments to be described below or to the configurations illustrated in the drawings, and includes configurations equivalent thereto.

The embodiments to be described below indicate comprehensive or specific examples. Numerical values, shapes, materials, constituent elements, positions and connection methods of the constituent elements, steps, and the sequence of the steps are examples and are not intended to limit the scope of the claims. Furthermore, the drawings are not necessarily exact illustrations. In each drawing, same reference signs are given to substantially identical structures, and redundant descriptions may sometimes be omitted or simplified.

In the following description, terms, such as parallel and orthogonal, expressing the relationships between elements, terms, such as cylindrical, expressing the shapes of elements, and numerical ranges do not express exact meanings, and may include a substantially equivalent range, such as a range having an error of about several %.

### Embodiment 1

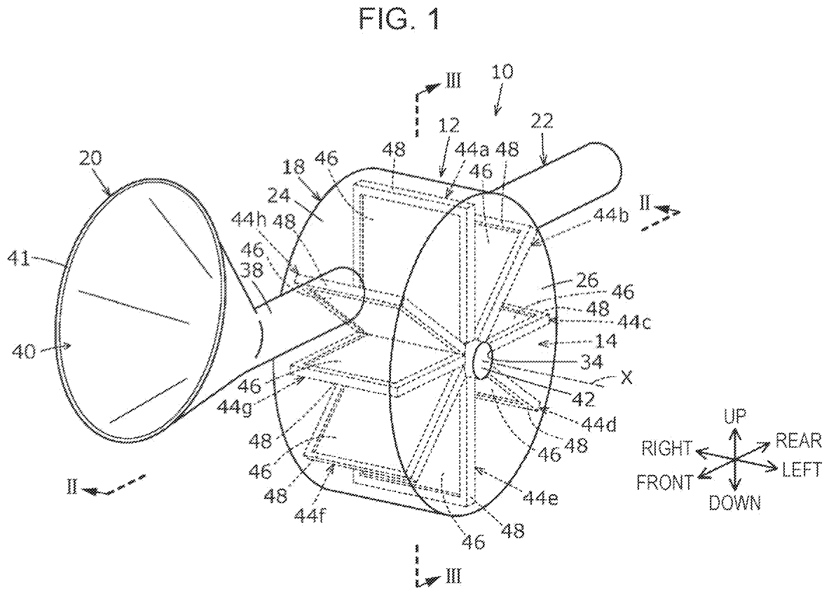

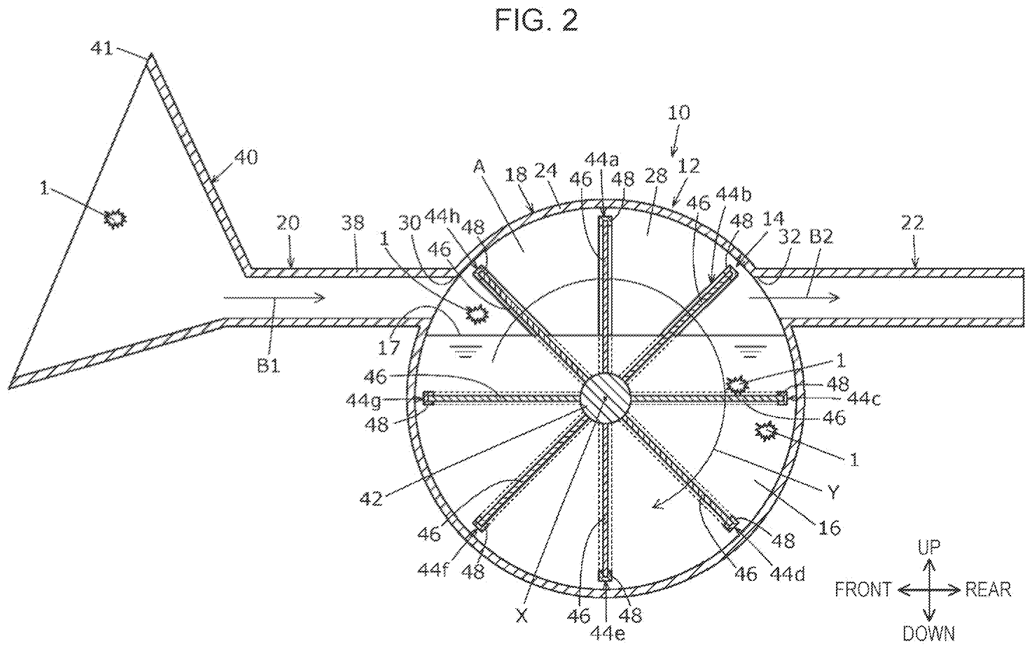

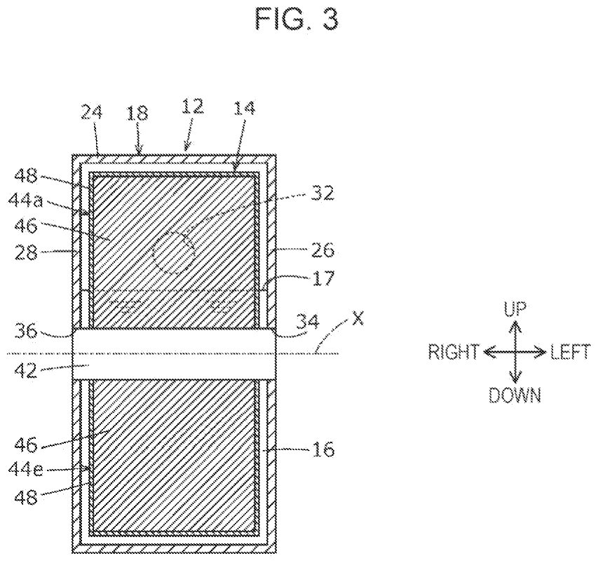

FIG. 1 is a perspective view illustrating a collection device 10 according to Embodiment 1. FIG. 2 is a sectional view taken along line II-II in FIG. 1. FIG. 3 is a sectional view taken along line III-III in FIG. 1. The collection device 10 according to Embodiment 1 will be described with reference to FIGS. 1 to 3. In FIG. 1, a collection liquid 16 (to be described later) is not shown for avoiding a complex illustration. In FIG. 3, first blades 44b and 44d (to be described later) are not shown for avoiding a complex illustration. Furthermore, in this embodiment, the side with a gas inlet 20 (to be described later) is described as the front side and the side with a gas outlet 22 (to be described later) is described as the rear side (see arrows in FIGS. 1 and 2), as viewed from a peripheral wall 24 (to be described later) of a main body 18 (to be described later). Moreover, in this embodiment, the side with a left wall 26 (to be described later) is described as the left side and the side with a right wall 28 (to be described later) is described as the right side (see arrows in FIGS. 1 and 3), as viewed from the peripheral wall 24. Furthermore, in this embodiment, the side with a channel A is described as the upper side and the side opposite the channel A is described as the lower side, as viewed from a shaft 42 (to be described later) (see arrows in FIGS. 1 and 3). The front-rear direction, the left-right direction, and the up-down direction intersect orthogonally to one another.

As shown in FIGS. 1 to 3, the collection device 10 collects a target object 1 in intake gas into a liquid. For example, the target object 1 includes a pathogen, a virus, a pollen, and particulate matter. The collection device 10 includes a container 12, a rotating body 14, and a collection liquid 16. Each of these structures will be described below.

The container 12 retains the collection liquid 16 inside the container 12 and has a channel A through which gas travels above the collection liquid 16 retained. The container 12 supports the rotating body 14 in a rotatable manner around a rotation axis X inside the container 12 (see an arrow Y in FIG. 2). The rotation axis X extends in the left-right direction. In other words, the rotation axis X extends orthogonally to the up-down direction. The rotation axis X does not necessarily have to extend orthogonally to the up-down direction as long as the rotation axis X extends in a direction intersecting the up-down direction. The container 12 has a main body 18, a gas inlet 20, and a gas outlet 22.

The main body 18 is a portion where the collection liquid 16 is retained and where the rotating body 14 is rotatably supported around the rotation axis X. The main body 18 is cylindrical and has a peripheral wall 24, a left wall 26, and a right wall 28.

The peripheral wall 24 is disposed around the rotation axis X, extends in the left-right direction, and is cylindrical. The peripheral wall 24 is provided to surround the rotating body 14. The peripheral wall 24 has a hole 30 and a hole 32 that are located above the rotation axis X and that extend through the peripheral wall 24 in the front-rear direction. The hole 30 is provided at the front side of the rotation axis X, whereas the hole 32 is provided at the rear side of the rotation axis X. The hole 30 and the hole 32 face each other in the front-rear direction. The left wall 26 and the right wall 28 extend orthogonally to the rotation axis X and are substantially disk-shaped. The left wall 26 and the right wall 28 face each other in the left-right direction. The left wall 26 is connected to the left edge of the peripheral wall 24 to cover the rotating body 14 from the left side. The left wall 26 has a circular hole 34 extending in the left-right direction through the center of the left wall 26. The right wall 28 is connected to the right edge of the peripheral wall 24 to cover the rotating body 14 from the right side. The right wall 28 has a circular hole 36 extending in the direction of the rotation axis X through the center of the right wall 28.

The gas inlet 20 takes in exhaled air of a user using the collection device 10 or gas, such as air, surrounding the collection device 10, and is connected to the main body 18. The user is, for example, a patient carrying a pathogen. The gas inlet 20 is tubular and has a cylindrical section 38 and a conical tubular section 40. The cylindrical section 38 extends in the front-rear direction and is cylindrical. The cylindrical section 38 is connected to the peripheral wall 24 so as to communicate with the hole 30. The conical tubular section 40 gradually widens toward the front side and is conical and tubular. The conical tubular section 40 is connected to an end of the cylindrical section 38 opposite from the peripheral wall 24.

The gas outlet 22 discharges the gas taken in from the gas inlet 20 and is connected to the main body 18 at the opposite side from the gas inlet 20. The gas outlet 22 extends in the front-rear direction and is cylindrical. The gas outlet 22 is connected to the peripheral wall 24 so as to communicate with the hole 32.

The container 12 has the channel A constituted by the gas inlet 20, the main body 18, and the gas outlet 22. The gas taken into the container 12 flows through the channel A. The gas taken into the gas inlet 20 travels through the conical tubular section 40 and the cylindrical section 38 (see an arrow B1 in FIG. 2), and flows into the main body 18 from the hole 30. The gas flowing into the main body 18 travels above the collection liquid 16 and flows into the gas outlet 22 from the hole 32 (see an arrow B2 in FIG. 2). Then, the gas is discharged from the gas outlet 22. Accordingly, the container 12 has the channel A above the collection liquid 16 such that the gas flows in the front-rear direction. In other words, when viewed from the up-down direction, the container 12 has the channel A above the collection liquid 16 such that the gas flows orthogonally to the rotation axis X. The container 12 does not necessarily have to have the channel A such that the gas flows orthogonally to the rotation axis X when viewed from the up-down direction. For example, the container 12 may have the channel A such that the gas flows in a direction intersecting the rotation axis X when viewed from the up-down direction.

The rotating body 14 is provided inside the container 12 and is supported by the container 12 in a rotatable manner around the rotation axis X. The rotating body 14 has a shaft 42 and first blades 44a to 44h.

The shaft 42 extends in the left-right direction and is columnar. The shaft center of the shaft 42 is aligned with the rotation axis X. The shaft 42 is provided at the cylindrical center of the main body 18. In detail, the left end of the shaft 42 is rotatably fitted in the hole 34 of the left wall 26, and the right end of the shaft 42 is rotatably fitted in the hole 36 of the right wall 28. Accordingly, the shaft 42 is supported by the main body 18 in a rotatable manner around the rotation axis X.

The first blades 44a to 44h protrude in the direction intersecting the rotation axis X and are attached to the outer peripheral surface of the shaft 42 at equal intervals around the rotation axis X. In other words, the first blades 44a to 44h protrude outward in the radial direction of the shaft 42 from the outer peripheral surface of the shaft 42 and are arranged at equal intervals in the circumferential direction of the shaft 42. The first blades 44a to 44h are rotatable together with the shaft 42 around the rotation axis X. The first blade 44a has a filter 46 and a frame 48.

The filter 46 extends in the left-right direction and in the direction intersecting the rotation axis X, and has a rectangular plate shape. The filter 46 does not necessarily have to have a rectangular plate shape. The filter 46 protrudes in the direction intersecting the rotation axis X and is connected to the outer peripheral surface of the shaft 42. When the rotating body 14 rotates around the rotation axis X, the filter 46 rotates around the rotation axis X. For example, when the filter 46 is in contact with the gas, the filter 46 captures the target object 1 in the gas. Moreover, for example, the filter 46 allows the gas to permeate therethrough, and captures the target object 1 in the permeated gas. The filter 46 has, for example, a net-like structure. The filter 46 used may be any one of various known filters that can capture the target object 1 in the gas. For example, a filter that uses a nonwoven fabric may be used as the filter 46. For example, a filter having porosity that allows the gas to permeate therethrough when the gas flows through the channel A and that causes the rotating body 14 to rotate around the rotation axis X by receiving the gas may be used as the filter 46. When the filter 46 is located directly above the rotation axis X (i.e., at the position of the first blade 44a in FIG. 2), the filter 46 protrudes upward from a liquid surface 17 of the collection liquid 16 and is located inside the channel A. In detail, when the filter 46 is located directly above the rotation axis X, part of the filter 46 is in the channel A, whereas the remaining part of the filter 46 is immersed in the collection liquid 16. Moreover, when the filter 46 is located directly above the rotation axis X, the filter 46 overlaps the hole 30 and the hole 32 in the front-rear direction. On the other hand, when the filter 46 is located directly below the rotation axis X (i.e., at the position of the first blade 44e in FIG. 2) and also when the filter 46 is located directly beside the rotation axis X (i.e., at the position of the first blade 44c or the position of the first blade 44g in FIG. 2), the filter 46 is located below the liquid surface 17 of the collection liquid 16 and is immersed in the collection liquid 16. In detail, when the filter 46 is located directly below the rotation axis X and also when the filter 46 is located directly beside the rotation axis X, the filter 46 is entirely immersed in the collection liquid 16. When the rotating body 14 rotates around the rotation axis X, the filter 46 is movable from a position inside the channel A to a position immersed in the collection liquid 16. In detail, the filter 46 rotates around the rotation axis X along with rotation of the rotating body 14 around the rotation axis X and is movable from the position inside the channel A to the position immersed in the collection liquid 16. In more detail, when the rotating body 14 rotates around the rotation axis X, part of the filter 46 existing in the channel A is immersed in the collection liquid 16.

The frame 48 has an opening at the shaft 42 side, is substantially U-shaped, and is provided along the edges of the filter 46. The frame 48 is attached to the outer peripheral surface of the shaft 42. The frame 48 is provided so as to clamp the edges of the filter 46. The frame 48 is separated from the inner surface of the main body 18 by a gap. In detail, in the direction orthogonal to the rotation axis X, the frame 48 is separated from the inner surface of the peripheral wall 24 by a gap. Furthermore, in the left-right direction, the frame 48 is separated from the inner surface of the left wall 26 by a gap, and is also separated from the inner surface of the right wall 28 by a gap.

Because the first blades 44b to 44h each have a configuration similar to that of the first blade 44a, a detailed description of the first blades 44b to 44h will be omitted by referring to the above description of the first blade 44a.

The collection liquid 16 is a liquid for collecting the target object 1 and is contained inside the main body 18. In detail, the collection liquid 16 is contained inside the main body 18 such that the liquid surface 17 of the collection liquid 16 is formed above the rotation axis X and above the shaft 42. Moreover, the collection liquid 16 is contained inside the main body 18 such that the liquid surface 17 of the collection liquid 16 is formed below the hole 30 and the hole 32. Accordingly, the collection liquid 16 may be injected to have a height at which the collection liquid 16 does not overflow from the gas inlet 20 and the gas outlet 22.

Next, a method for collecting the target object 1 in the gas by using the collection device 10 according to Embodiment 1 will be described. The following description relates to a method for collecting the target object 1 in exhaled air of a patient.

First, the collection device 10 is attached to the patient. In detail, the collection device 10 is attached to the patient such that the patient's mouth is covered with the conical tubular section 40 of the gas inlet 20. When the collection device 10 is attached to the patient, if there is a gap between the conical tubular section 40 and the area around the patient's mouth, the exhaled air of the patient may leak outside, possibly making it difficult to sufficiently collect a small amount of pathogen existing in the exhaled air of the patient. Therefore, a polyurethane sheet in the form of tape may be bonded to an opening 41 at the front side of the conical tubular section 40, so as to fill in the gap between the conical tubular section 40 and the area surrounding the patient's mouth. Furthermore, in order to make the collection device 10 easily attachable to the patient, the section, such as the cylindrical section 38, that connects the conical tubular section 40 and the main body 18 may be a rubber tube having flexibility. Accordingly, the orientation of the conical tubular section 40 can be readily changed, and the patient can send exhaled air into the main body 18 of the collection device 10 more reliably without taking an uncomfortable position.

After the collection device 10 is attached to the patient, the patient inhales through the nose to take in air into the lungs and subsequently exhales through the mouth, thereby blowing exhaled air into the main body 18 via the gas inlet 20. Although not specifically described here, it is also effective to use a device, such as a flowmeter, to check whether a required amount of exhaled air is taken into the container 12, and to prompt the patient to blow in exhaled air again if the required amount is not reached. The amount of exhaled air required for an examination may be calculated in advance, and the amount of exhaled air required for the examination can be taken into the container 12 more reliably in accordance with the calculation result.

As mentioned above, the main body 18 preliminarily contains the collection liquid 16. The collection liquid 16 used may be, for example, a physiological saline solution so that the pathogen to be collected is less likely to be damaged. The amount of the collection liquid 16 is, for example, about 5 mL. Since the collection liquid 16 may possibly leak from the main body 18 if the main body 18 is significantly tilted toward the gas inlet 20 or the gas outlet 22, the main body 18 may be fixed in a non-tiltable manner. The collection liquid 16 may contain a surfactant.

When the patient blows in exhaled air, the wind pressure of the exhaled air blown in by the patient causes the rotating body 14 to rotate around the rotation axis X, and the exhaled air passes through the filters 46 of the rotating body 14. With regard to each filter 46, a filter having porosity that causes the filter to receive resistance or less force from the wind pressure of the exhaled air when the exhaled air is blown against the filter 46 is selected, and thus the rotating body 14 can be readily rotated by the exhaled air.

As will be described below in Embodiment 2, in order to cause the rotating body to rotate more reliably by the exhaled air, first blades 50a to 50h (to be described later) of a rotating body 14a (to be described later) may have a flexible member composed of silicone in a section (i.e., contact section 51 (to be described later)) that is in contact with the inner surface of a main body 18a constituting the channel A. The flexible member is adjusted and set to a position where the flexible member can prevent the rotation of the rotating body 14a from stopping while also preventing the rotational driving force of the rotating body 14a from being lost due to the exhaled air leaking from a gap between the rotating body 14a and the main body 18a.

Furthermore, as will be described below in Embodiment 3, in order to obtain the rotational driving force of the rotating body more reliably, any of the first blades 44a to 44h may be changed to a Teflon (registered trademark) plate member (second blades 52a, 52c, 52e, and 52g (to be described later)) that is less gas-permeable than the filter 46.

The exhaled air blown in by the patient causes the rotating body 14 to rotate. While sequentially coming into contact with the filters 46, the exhaled air flows toward the gas outlet 22. Furthermore, when the rotating body 14 rotates, the first blades 44a to 44h sequentially move into the channel A and the exhaled air can come into contact with the first blades 44a to 44h more readily, whereby the rotating body 14 can be rotated more readily. The target object 1, such as a pathogen, contained in the exhaled air adheres to the surface of the filter 46. When the filter 46 is immersed in the collection liquid 16 as a result of the rotation of the rotating body 14, the target object 1, such as a pathogen, is isolated and collected by the collection liquid 16.

Accordingly, in a case where the intake gas is the exhaled air of the patient, the rotating body 14 disposed inside the main body 18 rotates when the patient blows the exhaled air into the main body 18. In this case, if the exhaled air of the patient contains the target object 1, such as a pathogen, the target object 1, such as a pathogen, is captured by the surface of the filter 46 of the rotating body 14 when the exhaled air permeates through or comes into contact with the filter 46. Along with the rotation of the rotating body 14, the captured target object 1, such as a pathogen, comes into contact with the collection liquid 16 and is removed from the filter 46, so as to become isolated and collected by the collection liquid 16.

The target object 1 in the gas can be thus collected into the liquid.

Finally, the collection liquid 16 that has collected the target object 1 is recovered, and is examined for the presence or absence of a pathogen by using a pathogen detector. If the recovered collection liquid 16 contains a pathogen, the pathogen can be extracted by using the pathogen detector. The collection device 10 may be connected to the pathogen detector, and the series of operation from the process for taking in the exhaled air of the patient to the process for examining the presence or absence of the pathogen can be automated.

As described above, the collection device 10 according to this embodiment collects the target object 1 in intake gas into the collection liquid 16, and includes the container 12 that retains the collection liquid 16 and that has the channel A through which the intake gas travels above the collection liquid 16 retained, and also includes the rotating body 14 that is provided inside the container 12 and that rotates around the rotation axis X extending in the direction intersecting the up-down direction. The rotating body 14 has the first blades 44a to 44h that protrude in the direction intersecting the rotation axis X. The first blades 44a to 44h each have the filter 46 that captures the target object 1 in the intake gas. The filter 46 moves, along with rotation of the rotating body 14 around the rotation axis X, from the position inside the channel A to the position immersed in the collection liquid 16.

Accordingly, the first blades 44a to 44h each have the filter 46 that captures the target object 1 in the gas, and the filter 46 moves, along with rotation of the rotating body 14 around the rotation axis X, from the position inside the channel A to the position immersed in the collection liquid 16. Therefore, from the state where the filter 46 is disposed inside the channel A, the filter 46 is immersed in the collection liquid 16 by rotating the rotating body 14 around the rotation axis X, so that the target object 1 in the gas inside the channel A can be captured, and the captured target object 1 can be collected into the collection liquid 16. In this way, when the filter 46 is rotated around the rotation axis X, the target object 1 in the gas can be easily collected.

### Embodiment 2

Next, Embodiment 2 will be described. Embodiment 2 is mainly different from Embodiment 1 in that a rotating body 14a rotates around the rotation axis X in a state where an edge of a first blade 50a (50b to 50h) is in contact with the inner surface of a container 12a that constitutes the channel A.

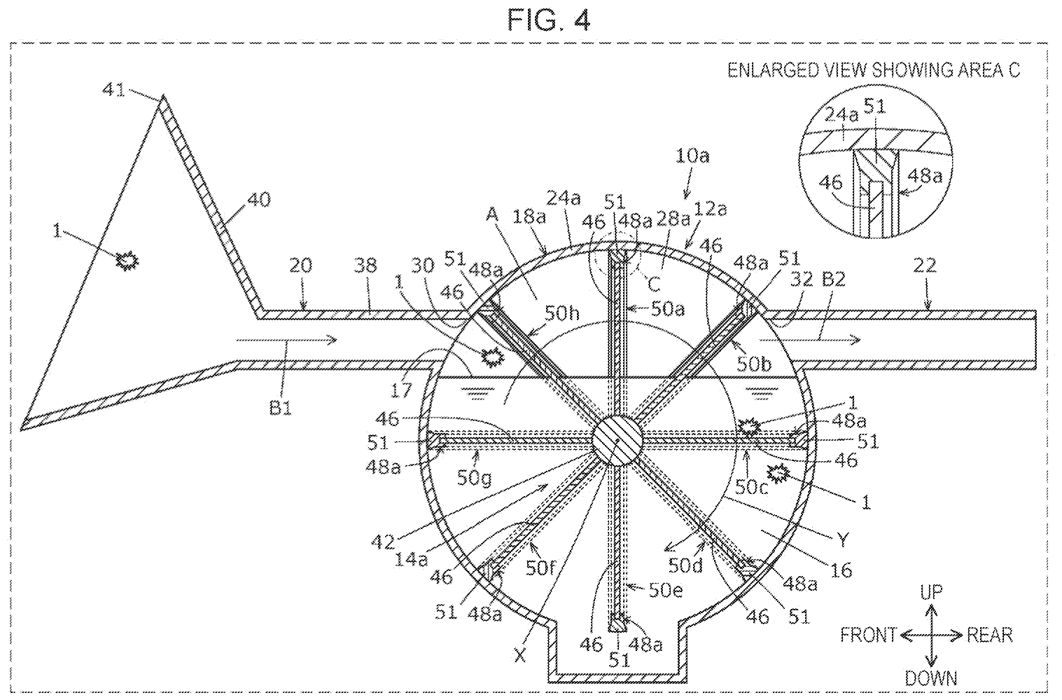

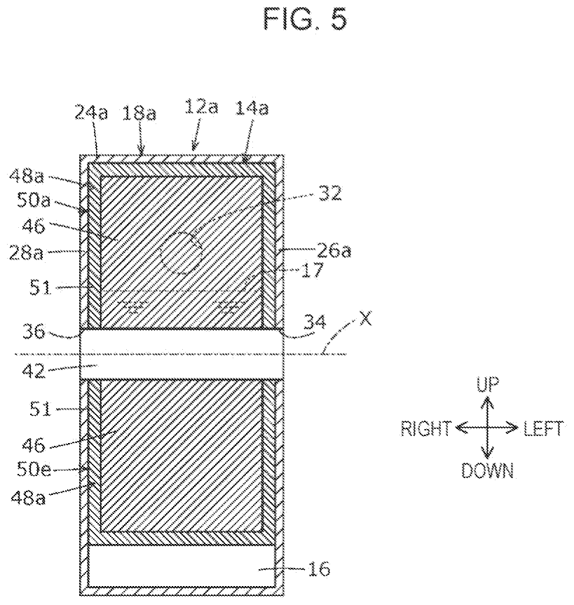

FIG. 4 is a sectional view illustrating a collection device 10a according to Embodiment 2, as viewed from the left. FIG. 5 is a sectional view illustrating the collection device 10a according to Embodiment 2, as viewed from the front. The collection device 10a according to Embodiment 2 will be described with reference to FIGS. 4 and 5. The following description mainly focuses on the differences from the collection device 10 according to Embodiment 1.

The collection device 10a is different from the collection device 10 in that the collection device 10a has a main body 18a different from the main body 18 and first blades 50a to 50h different from the first blades 44a to 44h. The main body 18a is different from the main body 18 in that the lower section of the main body 18a is recessed downward. This makes it easier to gather the target object 1 in the collection liquid 16 to the lower section of the main body 18a. The first blade 50a is different from the first blades 44a to 44h in that the first blade 50a has a frame 48a different from the frame 48. The frame 48a has a contact section 51 that is in contact with the inner surface of the main body 18a of the container 12a constituting the channel A. The inner surface of the main body 18a constituting the channel A refers to part of the inner surface of the main body 18a that is located above the liquid surface 17. The contact section 51 is provided at the outer edge of the frame 48a. The contact section 51 gradually widens outward and is in contact with the inner surface of the main body 18a. In detail, in the direction orthogonal to the rotation axis X, the contact section 51 gradually widens outward and is in contact with the inner surface of a peripheral wall 24a constituting the channel A. Moreover, in the left-right direction, the contact section 51 gradually widens leftward and is in contact with the inner surface of a left wall 26a constituting the channel A. Furthermore, in the left-right direction, the contact section 51 gradually widens rightward and is in contact with the inner surface of a right wall 28a constituting the channel A. For example, a flexible member composed of silicone may be used as the contact section 51. The rotating body 14a according to Embodiment 2 rotates around the rotation axis X in a state where the edge (i.e., the contact section 51) of the first blade 50a is in contact with the inner surface of the main body 18a constituting the channel A. Because the first blades 50b to 50h each have a configuration similar to that of the first blade 50a, a detailed description of the first blades 50b to 50h will be omitted by referring to the above description of the first blade 50a.

As described above, in the collection device 10a according to this embodiment, the rotating body 14a rotates around the rotation axis X in a state where the edge (i.e., the contact section 51) of the first blade 50a (50b to 50h) is in contact with the inner surface of the container 12a constituting the channel A.

Consequently, the rotating body 14a rotates around the rotation axis X in a state where the edge (i.e., the contact section 51) of the first blade 50a (Sob to 50h) is in contact with the inner surface of the main body 18a of the container 12a constituting the channel A. By providing the edge of each of the first blades 50a to 50h with the contact section 51 in this manner, the gas flowing through the channel A can be prevented from flowing through a gap between the main body 18a and the rotating body 14a. Thus, the gas flowing through the channel A can easily permeate through the filter 46, so that the target object 1 in the gas can be captured more reliably. In other words, a situation where the gas flowing through the channel A is discharged without permeating through the filter 46 can be suppressed. As an alternative to Embodiment 2 shown in FIGS. 4 and 5 in which the contact section 51 is provided along the edges of the frame 48a in the direction orthogonal to the rotation axis X and in the left-right direction, the contact section 51 may be provided at the edge of the frame 48a in the direction orthogonal to the rotation axis X, or the contact section 51 may be provided at the edges of the frame 48a in the left-right direction, as long as the contact section 51 is provided so as not to inhibit the rotation of the rotating body 14a. Furthermore, the contact section 51 does not necessarily have to gradually widen outward.

### Embodiment 3

Next, Embodiment 3 will be described. Embodiment 3 is mainly different from Embodiment 1 in that second blades 52a, 52c, 52e, and 52g are provided in place of the first blades 44a, 44c, 44e, and 44g.

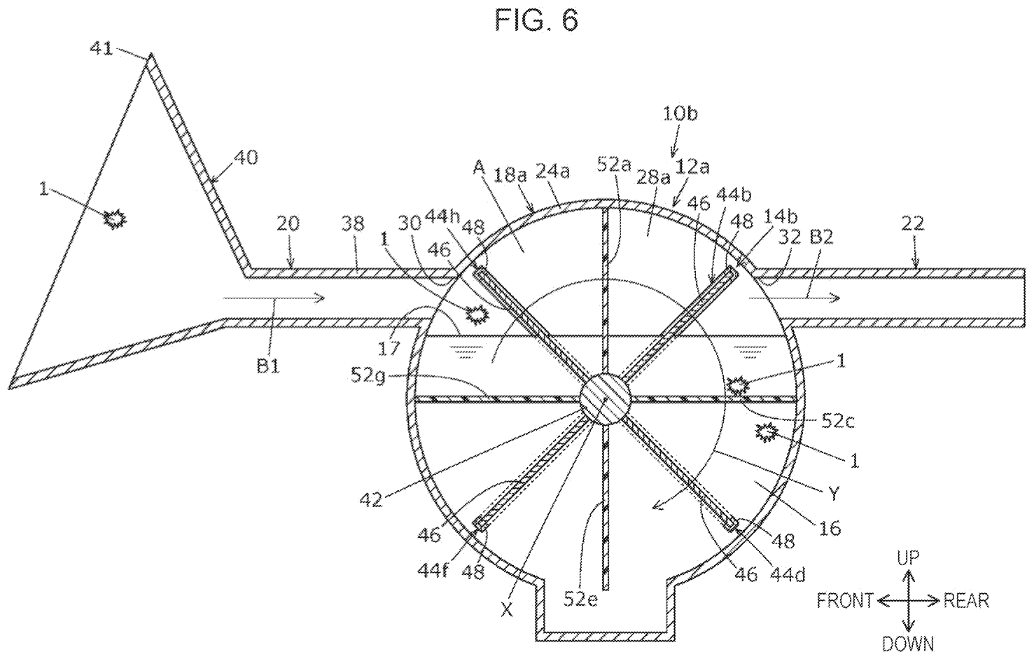

FIG. 6 is a sectional view illustrating a collection device 10b according to Embodiment 3, as viewed from the left. The collection device 10b according to Embodiment 3 will be described with reference to FIG. 6. The following description mainly focuses on the differences from the collection device 10 according to Embodiment 1.

The collection device 10b is different from the collection device 10 according to Embodiment 1 in that the collection device 10b has the container 12a in place of the container 12. Furthermore, the collection device 10b is different from the collection device 10 according to Embodiment 1 in that the collection device 10b has a rotating body 14b in place of the rotating body 14. A detailed description of the container 12a will be omitted by referring to the above description of Embodiment 2. The rotating body 14b is different from the rotating body 14 in that the rotating body 14b has the second blade 52a in place of the first blade 44a, the second blade 52c in place of the first blade 44c, the second blade 52e in place of the first blade 44e, and the second blade 52g in place of the first blade 44g. The second blades 52a, 52c, 52e, and 52g protrude in the direction intersecting the rotation axis X and are arranged at equal intervals around the rotation axis X. In other words, the second blades 52a, 52c, 52e, and 52g protrude outward in the radial direction of the shaft 42 from the outer peripheral surface of the shaft 42 and are attached to the outer peripheral surface of the shaft 42. The second blade 52a extends in the left-right direction and in the direction orthogonal to the rotation axis X, and has a substantially rectangular plate shape. The second blade 52a is in contact with the inner surface of the main body 18a of the container 12a constituting the channel A. In detail, an edge of the second blade 52a in the direction orthogonal to the rotation axis X is in contact with the inner surface of the peripheral wall 24a constituting the channel A. Furthermore, an edge of the second blade 52a in the left-right direction is in contact with the inner surface of the left wall 26a (see FIG. 5) constituting the channel A. Moreover, another edge of the second blade 52a in the left-right direction is in contact with the inner surface of the right wall 28a constituting the channel A. The second blade 52a is disposed along with the first blades 44b, 44d, 44f, and 44h around the rotation axis X. When the rotating body 14b rotates around the rotation axis X, the second blade 52a rotates around the rotation axis X. The rotating body 14b rotates around the rotation axis X in a state where the edges of the second blade 52a are in contact with the inner surface of the main body 18a constituting the channel A. The second blade 52a may be made of resin, such as Teflon (registered trademark), and the second blade 52a is less gas-permeable than the filter 46. The second blade 52a may be non-gas-permeable. Because the second blades 52c, 52e, and 52g each have a configuration similar to that of the second blade 52a, a detailed description of the second blades 52c, 52e, and 52g will be omitted by referring to the above description of the second blade 52a.

The first blade 44b (44d, 44f, 44h) may be in contact with the inner surface of the main body 18a of the container 12a constituting the channel A. In this case, the rotating body 14b rotates around the rotation axis X in a state where the first blade 44b (44d, 44f, 44h) is in contact with the inner surface of the main body 18a of the container 12a constituting the channel A.

As describe above, in the collection device 10b according to this embodiment, the rotating body 14b further has the second blades 52a, 52c, 52e, and 52g that protrude in the direction intersecting the rotation axis X and that are less gas-permeable than the filters 46.

Consequently, the rotating body 14b has the second blades 52a, 52c, 52e, and 52g that are less gas-permeable than the filters 46. For example, if intake gas is exhaled air of a patient, the second blades 52a, 52c, 52e, and 52g that are less gas-permeable than the filters 46 make the rotating body 14b receive the force from the exhaled air more readily, so that the rotating body 14b can be easily rotated. Thus, the target object 1 in the gas can be collected into the collection liquid 16 more easily. The number of second blades is not limited as long as the rotating body 14b can be rotated by the exhaled air and the pathogen in the exhaled air can be captured by each filter 46.

### Embodiment 4

Next, Embodiment 4 will be described. Embodiment 4 is mainly different from Embodiment 1 in that a driver 54 is further provided.

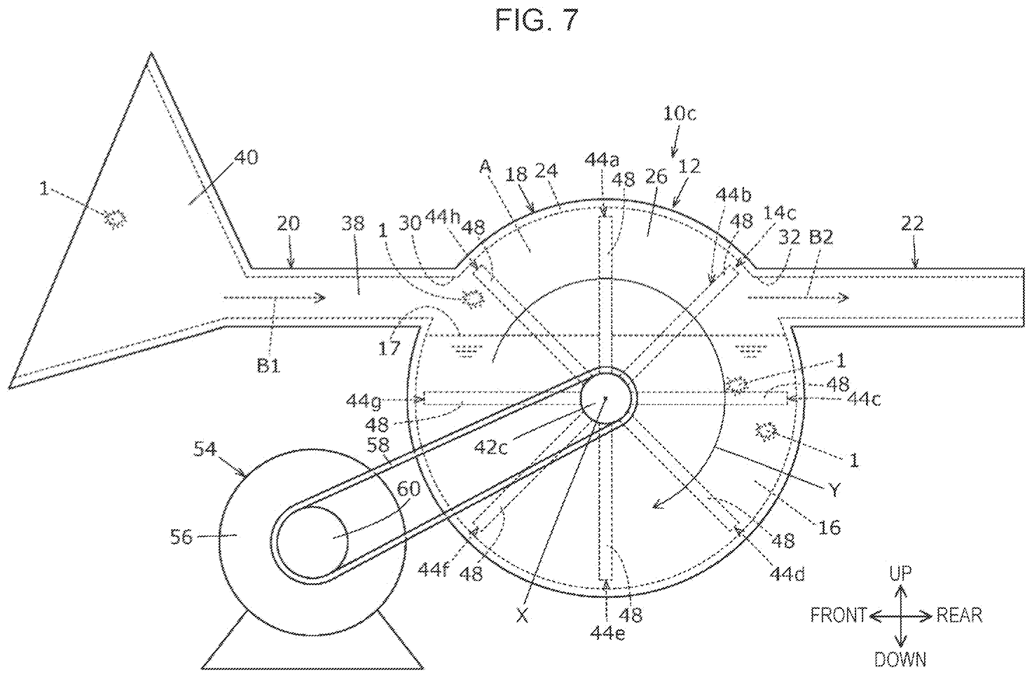

FIG. 7 is a side view illustrating a collection device 10c according to Embodiment 4, as viewed from the left. The collection device 10c according to Embodiment 4 will be described with reference to FIG. 7. The following description mainly focuses on the differences from the collection device 10 according to Embodiment 1.

The collection device 10c is different from the collection device 10 in that the collection device 10c further includes the driver 54. Furthermore, the collection device 10c is different from the collection device 10 in that a shaft 42c protrudes outward from the left wall 26 in the left-right direction. The driver 54 applies a rotational force to a rotating body 14c to rotate the rotating body 14c around the rotation axis X. The driver 54 has a motor 56 and a belt 58. The belt 58 is wrapped around the shaft 42c and a rotation shaft 60 of the motor 56. When the motor 56 is driven, the shaft 42c is rotated via the belt 58, whereby the rotating body 14c rotates. The rotating body 14c has the first blades 44a to 44h, but may alternatively have a single first blade.

As described above, the collection device 10c according to this embodiment further includes the driver 54 that rotates the rotating body 14c around the rotation axis X.

Thus, for example, if intake gas is indoor air, the shaft 42c of the rotating body 14c is linked with the motor 56 so that the rotation of the motor 56 is transmitted to the shaft 42c of the rotating body 14c, whereby the rotating body 14c can be easily rotated. Therefore, ambient air can be easily taken into the channel A, and the target object 1, such as a pathogen, contained in the ambient air can be easily isolated and collected into the collection liquid 16. By continuously rotating the rotating body 14c by using the driver 54, gas can be taken in efficiently, and the target object 1 in the gas can be collected efficiently.

In any of the embodiments described above with reference to FIGS. 1 to 7, after the target object, such as a pathogen, in the intake gas is captured by using the filter of the rotating body and is isolated and collected into the collection liquid with the rotation of the rotating body, the collection liquid may be extracted to be used for an examination of the pathogen. Furthermore, although not shown in the drawings, a pipe for extracting the collection liquid may be set at the lower section of the main body, and the collection liquid may be delivered by using, for example, a pump and be introduced to a pathogen examination device provided in combination with the collection device. By measuring the amount and the type of the pathogen in the collection liquid introduced to the pathogen examination device, an advantage of quickly preventing secondary infections of the pathogen can be expected.

### Embodiment 5

Next, Embodiment 5 will be described.

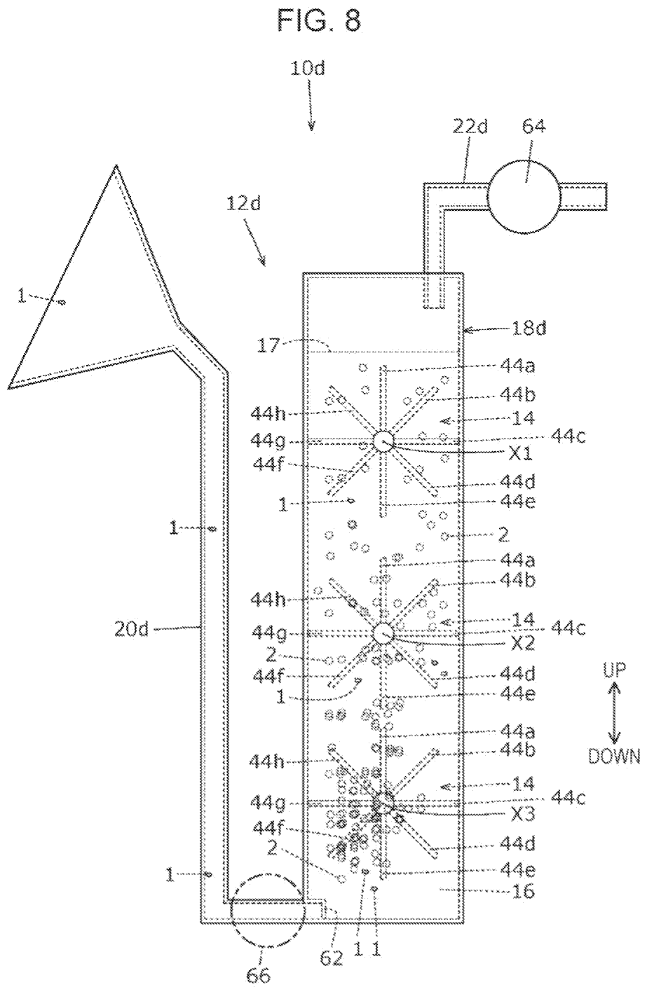

FIG. 8 is a side view illustrating a collection device 10d according to Embodiment 5. The collection device 10d according to Embodiment 5 will be described with reference to FIG. 8. In this embodiment, the side with a gas inlet 20d (to be described later) is referred to as the lower side and the side with a gas outlet 22d (to be described later) is referred to as the upper side, as viewed from a main body 18d (to be described later) (see arrows in FIG. 8).

As shown in FIG. 8, the collection device 10d collects a target object 1 in intake gas into a liquid. The collection device 16d includes a container 12d, three rotating bodies 14, and a collection liquid 16. Each of these structures will be described below.

The container 12d retains the collection liquid 16 inside the container 12d. The container 12d supports the three rotating bodies 14 in a rotatable manner around rotation axes X1, X2, and X3 extending orthogonally to the up-down direction. The rotation axes X1, X2, and X3 extend in the same direction and are arranged at intervals in the up-down direction. The rotation axes X1, X2, and X3 do not necessarily have to extend orthogonally to the up-down direction as long as they extend in a direction intersecting the up-down direction. The container 12d has a main body 18d, a gas inlet 20d, and a gas outlet 22d.

The main body 18d is a portion where the collection liquid 16 is retained and where the three rotating bodies 14 are rotatably supported around the rotation axes X1, X2, and X3. The main body 18d has a substantially rectangular parallelepiped shape and has an inflow port 62 through which the gas flows into the collection liquid 16 from below the rotating bodies 14. The inflow port 62 is provided at a lower section of the main body 18d. The inflow port 62 is, for example, a check valve that allows the gas to flow into the collection liquid 16 from the gas inlet 20d but does not allow the collection liquid 16 to flow to the gas inlet 20d.

The gas inlet 20d takes in exhaled air of a user using the collection device 10d or the air surrounding the collection device 10d, and is connected to the inflow port 62 of the main body 18d. The gas outlet 22d discharges the gas taken in from the gas inlet 20d and is connected to an upper section of the main body 18d. The gas outlet 22d communicates with a space above the collection liquid 16 inside the main body 18d.

The three rotating bodies 14 are immersed in the collection liquid 16 inside the container 12d and are arranged in the up-down direction. The rotating body 14 at the top is supported by the main body 18d in a rotatable manner around the rotation axis X1, the rotating body 14 in the middle is supported by the main body 18d in a rotatable manner around the rotation axis X2, and the rotating body 14 at the bottom is supported by the main body 18d in a rotatable manner around the rotation axis X3. A detailed description of the rotating bodies 14 will be omitted by referring to the above description in Embodiment 1. The filters 46 (see FIGS. 1 to 3) of the first blades 44a to 44h of each rotating body 14 come into contact with gas 2 flowing into the collection liquid 16 through the inflow port 62 and traveling upward in the collection liquid 16, so as to capture the target object 1 in the gas 2. When the gas 2 traveling in the collection liquid 16 comes into contact with the first blade 44a (44b to 44h), the rotating body 14 rotates around the rotation axis X1 (X2, X3). As a result of the rotation of the rotating body 14 around the rotation axis X1 (X2, X3), the target object 1 captured by the filter 46 is collected into the collection liquid 16. For example, the gas 2 flowing into the collection liquid 16 is in the form of bubbles.

The collection liquid 16 is a liquid for collecting the target object 1 and is contained inside the main body 18d. In detail, the collection liquid 16 is contained inside the main body 18d such that the liquid surface 17 of the collection liquid 16 is formed above the rotating body 14 at the top.

Although three rotating bodies 14 are disposed in the main body 18d of the collection device 10d, rotating bodies 14 are disposed for efficiently collecting a small amount of pathogen, and the number of rotating bodies 14 is not necessarily limited to three.

Furthermore, a phosphate buffer solution adjusted to a predetermined concentration may be injected as the collection liquid 16 into the main body 18d. The amount of phosphate buffer solution injected into the main body 18d may be such that all the rotating bodies 14 disposed therein are immersed in the solution, and the gas inlet 20d and the main body 18d may be connected to each other at a side surface of the lowermost section of the main body 18d or at the bottom thereof. The rotating bodies 14 may be disposed inside the main body 18d such that the gas entering through the gas inlet 20d efficiently comes into contact with the first blades 44a (44b to 44h) of the rotating bodies 14.

Furthermore, if the amount of pathogen in the gas is extremely small, multiple rotating bodies 14 may be set as shown in FIG. 8. In this case, the inflow port 62 is set at the lowermost section of the main body 18d that retains the collection liquid 16, so that the gas containing the pathogen comes into contact with the filters 46 of the rotating bodies 14 multiple times. During that time, the pathogen is captured by the filters 46 and is isolated and collected into the collection liquid 16 along with the rotation of the rotating bodies 14. In this case, a pump 64 provided at the gas outlet 22d located at the upper section of the main body 18d discharges the gas, so that the gas can be efficiently taken into the main body 18d from the gas inlet 20d.

Next, a method for collecting the target object 1 in the gas by using the collection device 10d according to Embodiment 5 will be described.

When the target object 1 in the exhaled air of the patient is to be collected, the patient blows the exhaled air into the gas inlet 20d so that the gas sequentially comes into contact with the first blades 44a (44b to 44h) of the rotating bodies 14 from the lower section while the target object 1, such as a pathogen, is captured by the filters 46. With the rotation of the rotating bodies 14, the target object 1, such as a pathogen, is isolated and collected into the phosphate buffer solution. If the target object 1, such as a pathogen, in the atmosphere is collected instead of the target object 1 in the exhaled air, for example, a pump 66 may be set at the gas inlet 20d of the collection device 10d. By actuating the pump 66, the gas is made to flow into the collection liquid 16. The type and the model of the pump 66 are not limited, as long as the pump 66 can take the target gas into the collection liquid 16. The phosphate buffer solution (i.e., the collection liquid 16) that has collected the target object 1, such as a pathogen, is recovered and is analyzed by using a pathogen detector. If the recovered phosphate buffer solution contains a pathogen, the pathogen can be extracted by using the pathogen detector.

As described above, the collection device 10d according to this embodiment collects the target object 1 in the intake gas into the collection liquid 16, and includes the container 12d that retains the collection liquid 16 and the rotating bodies 14 that are immersed in the collection liquid 16 inside the container 12d and that are each supported by the container 12d in a rotatable manner around the rotation axis X1 (X2, X3) extending in the direction intersecting the up-down direction. The container 12d has the inflow port 62 through which the gas flows into the collection liquid 16 from below the rotating bodies 14. Each rotating body 14 has the first blades 44a to 44h that protrude in the direction intersecting the rotation axis X1 (X2, X3). The first blades 44a to 44h have the filters 46 that capture the target object 1 in the gas by coming into contact with the gas flowing into the collection liquid 16 through the inflow port 62 and traveling upward in the collection liquid 16.

Accordingly, the gas flowing into the collection liquid 16 through the inflow port 62 provided below the rotating bodies 14 travels upward. Then, the filters 46 of the rotating bodies 14 come into contact with the gas traveling upward, so as to capture the target object 1 in the gas. Each rotating body 14 rotates around the rotation axis X1 (X2, X3) when the gas traveling in the collection liquid 16 comes into contact with the first blade 44a (44b to 44h). The rotating body 14 rotates around the rotation axis X1 (X2, X3) in this manner, so that the target object 1 captured by the filters 46 is collected into the collection liquid 16. By causing the gas to flow into the collection liquid 16 through the inflow port 62 in this manner, the target object 1 in the gas can be easily collected into the collection liquid 16. Furthermore, because the gas in the collection liquid 16 becomes small bubbles as a result of coming into contact with each rotating body 14 rotating around the rotation axis X1 (X2, X3), the gas can be readily captured by the filters 46. Thus, the target object 1 in the gas can be collected more reliably.

Although a collection device according to one or more aspects of the present disclosure has been described above with reference to the above embodiments, the present disclosure is not to be limited to these embodiments. An embodiment achieved by applying various modifications conceivable by a skilled person to any of the above embodiments or an embodiment configured by combining constituent elements in different embodiments may be included within the range of the one or more aspects of the present disclosure as long as the embodiment does not depart from the scope of the present disclosure.

In the above embodiments, the cylindrical section 38 and the gas outlet 22 extend in the front-rear direction. However, for example, the cylindrical section 38 may extend diagonally upward toward the front, and the gas outlet 22 may extend diagonally upward toward the rear.

In the above embodiments, the liquid surface 17 of the collection liquid 16 is formed above the rotation axis X. However, aspects of the present disclosure is not limited thereto. For example, the liquid surface 17 of the collection liquid 16 may be formed below the rotation axis X.

The present disclosure is widely applicable to a device that collects a target object in gas into a liquid.

What is claimed is:

1. A collection device comprising:
a container that retains a liquid and that has a channel through which intake gas travels above the liquid retained; and
a rotating body that is provided inside the container and that rotates around a rotation axis extending in a direction intersecting an up-down direction,
wherein the rotating body has a first blade that protrudes in a direction intersecting the rotation axis,
wherein the first blade has a filter that captures a target object included in the intake gas, and
wherein the filter moves, along with rotation of the rotating body around the rotation axis, from a position inside the channel to a position immersed in the liquid, thereby the collection device collecting, into the liquid, the target object included in the intake gas.

2. The collection device according to claim 1,
wherein the rotating body rotates around the rotation axis in a state where an edge of the first blade is in contact with an inner surface of the container constituting the channel.

3. The collection device according to claim 1,
wherein the rotating body further has a second blade that protrudes in the direction intersecting the rotation axis and that is less gas-permeable than the filter.

4. The collection device according to claim 1, further comprising:
a driver that causes the rotating body to rotate around the rotation axis.

5. A collection device comprising:
a container that retains a liquid; and
a rotating body that is immersed in the liquid inside the container and that rotates around a rotation axis extending in a direction intersecting an up-down direction,
wherein the container has an inflow port through which intake gas flows into the liquid from below the rotating body,
wherein the rotating body has a first blade that protrudes in a direction intersecting the rotation axis, and
wherein the first blade has a filter that captures a target object included in the intake gas by being in contact with the intake gas flowing into the liquid through the inflow port and traveling upward in the liquid, thereby the collection device collecting, into the liquid, the target object included in the intake gas.

6. A collection container comprising:
an inlet that receives exhaled air containing a target object;
a rotating body that has blades including a first blade having a first filter; and
a container that accommodates the rotating body and that has a first region filled with gas and a second region filled with a liquid,
wherein each of the blades rotates around an axis and is contact with the container, and
wherein the exhaled air causes the first blade to rotate and causes the first filter to move from the first region to the second region, and the target object captured by the first filter in the first region is released in the second region.

7. The collection container according to claim 6,
wherein the first filter is a nonwoven fabric provided on the first blade, and
wherein the liquid is a physiological saline solution.
